(19) United States
(12) Patent Application Publication
(10) Pub. No.: US 2024/0415496 A1
(43) Pub. Date: Dec. 19, 2024

(54) SYSTEM AND METHOD TO REGISTER AND CALIBRATE ULTRASOUND PROBE FOR NAVIGATION IN REAL TIME

(71) Applicant: Medtronic Navigation, Inc., Lafayette, CO (US)

(72) Inventors: Osvaldo Andres Barrera, Madison, CT (US); Joseph D. Brannan, Lyons, CO (US); Mark L. Stiger, Santa Rosa, CA (US); Anthony B. Ross, Boulder, CO (US); Bradley W. Jacobsen, Erie, CO (US); Patrick A. Helm, Canton, MA (US); Yvan R. Paitel, Louisville, CO (US); Kaustubh R. Patil, Plymouth, MN (US); Darion R. Peterson, Longmont, CO (US); Joshua J. Blauer, White Bear Township, MN (US); Trevor C. Huang, Maple Grove, MN (US)

(21) Appl. No.: 18/676,263

(22) Filed: May 28, 2024

Related U.S. Application Data
(60) Provisional application No. 63/521,144, filed on Jun. 15, 2023.

(51) Int. Cl. A61B 8/00

(52) U.S. Cl. CPC A61B 8/587

(57) ABSTRACT

A system includes an imaging system and a tracking system. The system generates a navigation space based on one or more tracking signals emitted by a transmission device. The system generates a virtual space including at least a portion of a calibration phantom based on one or more images generated by the imaging system. The system identifies a set of coordinates in the virtual space in response to an event in which at least a portion of the tracked device is detected in the one or more images. The system calibrates a first coordinate system associated with the virtual space with respect to a second coordinate system associated with the navigation space in response to the event. Calibrating the first coordinate system with respect to the second coordinate system is based on the set of coordinates and temporal information associated with the event.

### CROSS-REFERENCE TO RELATED APPLICATION

This application claims the benefit of and priority to U.S. Provisional Patent Application No. 63/521,144 filed Jun. 15, 2023, the entire disclosure of which is incorporated by reference herein.

### FIELD OF INVENTION

The present disclosure is generally directed to navigation and ultrasound imaging, and relates more particularly to calibrating an ultrasound probe for navigation.

### BACKGROUND

Imaging devices and navigation systems may assist a surgeon or other medical provider in carrying out a surgical procedure. Imaging may be used by a medical provider for visual guidance in association with diagnostic and/or therapeutic procedures. Navigation systems may be used for tracking objects (e.g., instruments, imaging devices, etc.) associated with carrying out the surgical procedure.

### BRIEF SUMMARY

Example aspects of the present disclosure include:

A system including: a processor; and a memory storing instructions that, when executed by the processor, cause the processor to: generate a navigation space based on one or more tracking signals; generate a virtual space including at least a portion of a calibration phantom based on one or more images, wherein the one or more images are generated in response to one or more signals transmitted by an imaging device; identify a set of coordinates in the virtual space in response to an event in which at least a portion of a tracked device in the calibration phantom is detected in the one or more images; and calibrate a first coordinate system associated with the virtual space with respect to a second coordinate system associated with the navigation space in response to the event, wherein calibrating the first coordinate system with respect to the second coordinate system is based on the set of coordinates and temporal information associated with the event.

Any of the aspects herein, wherein the set of coordinates are identified in the virtual space in response to at least the portion of the tracked device intersecting a surface of the virtual space at the set of coordinates.

Any of the aspects herein, wherein: calibrating the first coordinate system with respect to the second coordinate system is in response to one or more occurrences of the event; and calibrating the first coordinate system with respect to the second coordinate system is absent pausing a surgical procedure.

Any of the aspects herein, wherein calibrating the first coordinate system with respect to the second coordinate system is based on: beam thickness (e.g., ultrasound beam thickness), beam shape (e.g., ultrasound beam shape), or both of the one or more signals transmitted by the imaging device; pose information of the portion of the tracked device in association with an intersection between the portion of the tracked device and a plane of the virtual space; and one or more properties of the portion of the tracked device.

Any of the aspects herein, wherein the calibration phantom includes: ultrasound conductive material; or a tissue phantom included in a body of a subject, wherein the tissue phantom and the subject are associated with a surgical procedure.

Any of the aspects herein, wherein the instructions are further executable by the processor to: output guidance information associated with positioning the imaging device, the tracked device, or both in association with calibrating the first coordinate system with respect to the second coordinate system.

Any of the aspects herein, wherein the tracked device is included in at least a portion of an instrument, and the instructions are further executable by the processor to: detect, in the one or more images, one or more landmarks corresponding to at least a portion of the tracked device, wherein calibrating the first coordinate system with respect to the second coordinate system is based on the one or more landmarks.

Any of the aspects herein, wherein calibrating the first coordinate system with respect to the second coordinate system includes verifying a registration accuracy between the first coordinate system and the second coordinate system.

Any of the aspects herein, wherein the instructions are further executable by the processor to: detect one or more discrepancies between first tracking data corresponding to the tracked device in association with the navigation space and second tracking data corresponding to the tracked device in association with the virtual space; and generate a notification associated with the one or more discrepancies, perform one or more operations associated with compensating for the one or more discrepancies, or both.

Any of the aspects herein, wherein the virtual space corresponds to a field of view of the imaging device.

Any of the aspects herein, wherein the navigation space and the tracked device are associated with at least one of: an optical tracking system, an acoustic tracking system, an electromagnetic tracking system, a radar tracking system, a magnetic tracking system, an inertial measurement unit (IMU) based tracking system, and a computer vision based tracking system.

A system including: an imaging system including an imaging device; a tracking system including: a transmission device; and a tracked device; a calibration phantom; a processor; and a memory storing data that, when processed by the processor, cause the processor to: generate a navigation space based on one or more tracking signals emitted by the transmission device; generate a virtual space including at least a portion of the calibration phantom based on one or more images generated by the imaging system, wherein the one or more images are generated in response to one or more signals transmitted by the imaging device; identify a set of coordinates in the virtual space in response to an event in which at least a portion of the tracked device is detected in the one or more images; and calibrate a first coordinate system associated with the virtual space with respect to a second coordinate system associated with the navigation space in response to the event, wherein calibrating the first coordinate system with respect to the second coordinate system is based on the set of coordinates and temporal information associated with the event.

Any of the aspects herein, wherein the set of coordinates are identified in the virtual space in response to at least the portion of the tracked device intersecting a surface of the virtual space at the set of coordinates.

Any of the aspects herein, wherein: calibrating the first coordinate system with respect to the second coordinate system is in response to one or more occurrences of the event; and calibrating the first coordinate system with respect to the second coordinate system is absent pausing a surgical procedure.

Any of the aspects herein, wherein the calibration phantom includes: ultrasound conductive material; or a tissue phantom included in a body of a subject, wherein the tissue phantom and the subject are associated with a surgical procedure.

Any of the aspects herein, wherein calibrating the first coordinate system with respect to the second coordinate system is based on: beam thickness, beam shape, or both of the one or more signals transmitted by the imaging device; pose information of the portion of the tracked device in association with an intersection between the portion of the tracked device and a plane of the virtual space; and one or more properties of the portion of the tracked device.

A method including: generating a navigation space based on one or more tracking signals; generating a virtual space including at least a portion of a calibration phantom based on one or more images, wherein the one or more images are generated in response to transmitting one or more imaging signals; identifying a set of coordinates in the virtual space in response to an event in which at least a portion of a tracked device in the calibration phantom is detected in the one or more images; and calibrating a first coordinate system associated with the virtual space with respect to a second coordinate system associated with the navigation space in response to the event, wherein calibrating the first coordinate system with respect to the second coordinate system is based on the set of coordinates and temporal information associated with the event.

Any of the aspects herein, wherein the set of coordinates are identified in the virtual space in response to at least the portion of the tracked device intersecting a plane of the virtual space at the set of coordinates.

Any of the aspects herein, wherein: calibrating the first coordinate system with respect to the second coordinate system is in response to one or more occurrences of the event; and calibrating the first coordinate system with respect to the second coordinate system is absent pausing a surgical procedure.

Any of the aspects herein, wherein the calibration phantom includes: ultrasound conductive material; or a tissue phantom included in a body of a subject, wherein the tissue phantom and the subject are associated with a surgical procedure.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/implementations in combination with any one or more other aspects/features/implementations.

Use of any one or more of the aspects or features as disclosed herein.

It is to be appreciated that any feature described herein can be claimed in combination with any other feature(s) as described herein, regardless of whether the features come from the same described implementation.

The details of one or more aspects of the disclosure are set forth in the accompanying drawings and the description below. Other features, objects, and advantages of the techniques described in this disclosure will be apparent from the description and drawings, and from the claims.

The preceding is a simplified summary of the disclosure to provide an understanding of some aspects of the disclosure. This summary is neither an extensive nor exhaustive overview of the disclosure and its various aspects, implementations, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, implementations, and configurations of the disclosure are possible utilizing, alone or in combination, one or more of the features set forth above or described in detail below.

Numerous additional features and advantages of the present disclosure will become apparent to those skilled in the art upon consideration of the implementation descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, implementations, and configurations of the disclosure, as illustrated by the drawings referenced below.

FIG. 1A illustrates an example of a system in accordance with aspects of the present disclosure. FIG. 1B illustrates an example of a system in accordance with aspects of the present disclosure. FIG. 1C illustrates an example of a system in accordance with aspects of the present disclosure.

FIG. 2A illustrates an example implementation of a system in accordance with aspects of the present disclosure. FIG. 2B illustrates example aspects of a navigation space and a virtual space in accordance with aspects of the present disclosure.

FIG. 3A illustrates example views of an ultrasound beam in accordance with aspects of the present disclosure. FIG. 3B illustrates example implementations of an instrument in accordance with aspects of the present disclosure. FIG. 3C illustrates example implementations of an instrument in accordance with aspects of the present disclosure. FIG. 3D illustrates example aspects of an instrument in accordance with aspects of the present disclosure.

FIG. 4 illustrates an example of a process flow in accordance with aspects of the present disclosure.

FIG. 5 illustrates an example of a process flow in accordance with aspects of the present disclosure.

### DETAILED DESCRIPTION

It should be understood that various aspects disclosed herein may be combined in different combinations than the combinations specifically presented in the description and accompanying drawings. It should also be understood that, depending on the example or implementation, certain acts or events of any of the processes or methods described herein may be performed in a different sequence, and/or may be added, merged, or left out altogether (e.g., all described acts or events may not be necessary to carry out the disclosed techniques according to different implementations of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia Geforce RTX 2000-series processors, Nvidia Geforce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any implementations of the disclosure are explained in detail, it is to be understood that the disclosure is not limited in its application to the details of construction and the arrangement of components set forth in the following description or illustrated in the drawings. The disclosure is capable of other implementations and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

In some systems, an ultrasound probe may be calibrated/registered relative to a navigation means (e.g., a tracked sensor, etc.) in association with navigated image acquisition. For example, some systems may establish a transformation matrix that maps the six-dimensional (6D) pose (e.g., position and orientation information) of the tracked sensor to the 6D pose of an ultrasound probe. Some systems may map the 6D pose of the tracked sensor to an image generated by the ultrasound probe or to the ultrasound beam of the ultrasound probe.

In some cases, some calibration methods may be tedious, time consuming, and error prone. In some other cases, some calibration phantoms utilized for calibrating the ultrasound probe to the navigation system are costly and are prone to decay with time (e.g., due to the degradation of hydrogels implemented in some calibration phantoms).

Instances may occur in which a surgical team is unaware that the ultrasound probe has lost calibration with the navigation system. In some cases, even if the surgical team is aware of the loss in calibration, the team may be unwilling to recalibrate the ultrasound probe (e.g., due to lack of time or resources). Undetected or unaddressed loss in calibration during a medical procedure (e.g., due to deformation, tool drop/hit, etc.) may result in surgical errors. In some other cases, metal and other materials present in the environment may cause distortion to an electromagnetic field generated by the navigation system in association with tracking an object, and such distortion may result in surgical errors.

In accordance with aspects of the present disclosure, systems and techniques described herein may support dynamic initial calibration and dynamic recalibration of ultrasound probes (also referred to herein as ultrasonic probes) for navigation. The systems and techniques may incorporate an ultrasound probe connected to a main application/navigation system (also referred to herein as a navigated surgery system). The systems and techniques may include electromagnetic navigation of the ultrasound probe using trackers/sensors coupled to the ultrasound probe and an emitter capable of emitting electromagnetic signals. In some aspects, the systems and techniques may include an electromagnetic tracked device (e.g., an electromagnetic pointer (or stylus)) and a calibration phantom.

In some examples, the calibration phantom may be a phantom with a configuration of rods or wires. In some other examples, the calibration phantom may be a water bath or a tissue phantom, but is not limited thereto. The example calibration phantoms described herein are stable, inexpensive compared to some other calibration phantoms, and electromagnetic friendly. For example, the calibration phantoms may be free of materials that may interfere with electromagnetic navigation and tracking. In some example implementations, the calibration phantom may be a gel (e.g., hydrogel) for ultrasound calibration with optical tracking.

Examples of the techniques described herein may include moving or positioning a tracked device inside the calibration phantom while observing the movement of the tracked device using ultrasound imaging generated by an ultrasound imaging device, in which the ultrasound imaging corresponds to or represents an ultrasound view of the ultrasound imaging device. The techniques may include recording a video file of the ultrasound imaging concurrently with the tracking data and processing the video file (e.g., using a software script). Based on the processing of the video file, the techniques may include identifying a temporal instance at which a portion (e.g., tip) of the tracked device enters the ultrasound view. The systems and techniques may calibrate the ultrasound view with respect to the tracking space. Examples of the tracked device and the tracking space include an electromagnetic tracked device and an electromagnetic tracking space, but are not limited thereto. Aspects of the present disclosure support any type of tracked devices (e.g., sensors) and tracking spaces that may be implemented by a navigation system.

The systems and techniques described herein may support autonomous or semi-autonomous calibration, calibration verification with reduced calibration time compared to some other calibration techniques, and providing or outputting guidance information (e.g., tutorials, user prompts, etc.) for users on how to move or position a device (e.g., ultrasound imaging device, electromagnetic tracked device, an electromagnetic pointer (or stylus), etc.) in association with calibration. For example, the systems and techniques described herein support performing multiple calibrations (e.g., an initial calibration using a water bath, one or more subsequent calibrations using a tissue phantom, etc.), in which a system may perform the calibrations autonomously (or semi-autonomously). The systems and techniques may perform the calibrations continuously (or semi-continuously) and/or in response to trigger criteria, aspects of which are described herein. It is to be understood that as described herein with respect to calibration may be applied to recalibration. The terms “calibration,” “recalibration,” “calibration verification,” and “reregistration” may be used interchangeably herein.

Techniques described herein may be implemented in hardware, software, firmware, or any combination thereof that may automatically detect instrument landmarks on ultrasound images during a medical procedure. The techniques may include detecting landmarks of an instrument (e.g., tip of a needle during placement, distinctive features of navigated catheters, tip of a registration stylus, etc.) during the medical procedure and, using the detected instrument landmarks, automatically calibrating (or adjusting the calibration of) the ultrasound imaging device to the navigation system. The ultrasound imaging device may be, for example, an ultrasound probe.

Aspects of the automatic calibration techniques may provide a time savings for the surgical team, an improved user experience, and increased accuracy over longer portions of medical procedures. Other aspects of the calibration techniques provide cost savings through the use of, as a calibration phantom, an empty container (e.g., an empty box including an electromagnetic friendly material) with cross wires with patterns. The calibration phantom may be filled with water for the calibration procedure, thereby resulting in cost savings compared to other materials (e.g., gels, silicones, etc.). In some additional aspects, as water does not decay with time (e.g., compared to gels and silicones), using water in the calibration phantom may provide increased durability.

The electromagnetic tracking and calibration solutions described herein support directly using electromagnetic tools implemented in some existing medical procedures. In some aspects, direct use of such existing electromagnetic tools may provide increased accuracy due to accurate tracking of electromagnetic tools by some navigation systems.

In some examples, the calibration techniques described herein may be implemented using actual tissue of a subject as a calibration phantom. For example, the calibration techniques described herein may be implemented during a medical procedure associated with the actual tissue. In an example, if medical personnel is inserting a device (e.g., an ablation antenna, cardiac catheter, etc.) using ultrasound guidance provided by an ultrasound imaging device, the device will be visible in the ultrasound view. The calibration techniques and calibration software described herein may include using (e.g., automatically, or in response to a user request, etc.) the ultrasound images and corresponding electromagnetic tracking information to recalibrate the registration between the ultrasound space and the electromagnetic navigation space (also referred to herein as recalibrating the ultrasound tracking registration), without interrupting the medical procedure.

The systems and techniques described herein support recalibrating the ultrasound imaging system in the background based on automatic detection of target objects (e.g., instruments, tools, etc.) in the ultrasound images using AI/machine learning computer vision algorithms and object detection. The systems and techniques support automatic registration which may be implemented continuously, based on each event in which a tracked instrument or tracked device is detected in the ultrasound view, and/or periodically (e.g., based on a temporal trigger).

The systems and techniques support automatic registration in response to other trigger criteria (e.g., in response to detection of a target instrument in an ultrasound image) at any point during a medical procedure. In an example, the systems and techniques may include continuously verifying the registration accuracy between the ultrasound imaging system and the navigation system anytime the target instrument (e.g., surgical instrument, electromagnetic pointer, etc.) is detected in the ultrasound imaging. The systems and techniques support alerting the user and/or taking corrective actions in response to registration discrepancies. In an example, after outputting an alert (e.g., an audible alert, a visible alert, a haptic alert, etc.) to the user, the system may provide the user with a list of corrective actions for improving calibration. Non-limiting examples of corrective actions may include real-time actionable feedback for users to move the target instrument in association with registration.

In some other aspects, the systems and techniques may support dynamically and automatically detecting distortion in the navigated volume due to discrepancies between expected navigation and imaging data. In an example, the discrepancies may be between pose information of a tracked object as indicated by the navigation system and pose information of the tracked object as indicated by the imaging data. The systems described herein may support techniques for alerting (e.g., providing a notification to) a user of the discrepancies and compensating for the discrepancies. The systems and techniques may include calibrating the navigation data to the imaging data (e.g., calibrating a navigation space to an ultrasound space) while compensating for the discrepancies.

Aspects of the present disclosure support integration of a calibration phantom (e.g., water bath, hydrogel phantom, etc.) into the structure of a patient tracker. The integration of the calibration phantom may support user recalibration of a navigated ultrasound probe before or during a medical procedure. According to example aspects of the recalibration techniques described herein, the temporal duration associated with recalibration is reduced compared to some other recalibration techniques.

Implementations of the present disclosure provide technical solutions to one or more of the problems associated with other navigation systems and calibration techniques. For example, the systems and techniques described herein provide time savings, improved user experience, cost savings, and increased accuracy in comparison to other registration and calibration techniques. The systems and techniques described herein support continuous registration during a surgical procedure and continuous registration verification, in which registration and registration verification may be autonomous or semi-autonomous.

Aspects of the systems and techniques described herein support time efficient and cost-effective utilization of ultrasound images during surgery to navigate and display to medical personnel the locations of surgical devices (e.g., instruments, surgical tools, robotic end effectors, etc.) with respect to the patient anatomy. The systems and techniques described herein provide a reliable accuracy of the calibration between imaging devices (e.g., an ultrasound image probe, other imaging probes, etc.) and a navigation system, and the reliable accuracy may support accurate navigation of images (e.g., ultrasound images, etc.) that are generated based on data captured by the imaging devices.

In some aspects, different imaging probes may be different based on manufacturer, configuration, probe type, and the like, and such imaging probes may require new calibration and can lose calibration during a medical procedure. Aspects of the calibration techniques described herein are relatively time efficient, cost effective, user friendly, and provide increased accuracy compared to other techniques for calibrating or recalibrating imaging probes. The time efficiency, cost effectiveness, user friendliness, and increased accuracy supported by the systems and techniques described herein may provide improved confidence for a surgeon in a navigated ultrasound space and support a reduction in surgical errors. In some alternative and/or additional aspects, the registration and calibration techniques described herein may be implemented autonomously (e.g., without input from medical personnel) or semi-autonomously (e.g., with partial input from medical personnel).

Aspects of the present disclosure relate to navigated and robotic surgery and to any type of surgery that may be associated with intra-surgical ultrasound imaging. Aspects of the present disclosure support implementing any of the techniques described herein to any medical procedure (e.g., cranial, spinal, thoracic, abdominal, cardiac, ablation, laparoscopic, minimally invasive surgery, robotic surgery, etc.) associated with the use of intra-surgical ultrasound imaging. In some other aspects, the systems and techniques described herein may be implemented in association with initiatives related to data analytics, artificial intelligence, and machine learning, for example, with respect to data analytic scenarios for procedure and device optimization.

In some cases, the techniques described herein may be implemented as a standalone application that uses a calibration phantom (e.g., a water bath, etc.) and an imaging system (e.g., ultrasound imaging, optical or electromagnetic tracking, 3D rendering software, and calibration software) or as an application integrated with an imaging system or navigation system. The examples described herein with reference to the following figures may support multiple types, geometries, configurations, and sizes of calibration phantoms other than the examples illustrated and described herein.

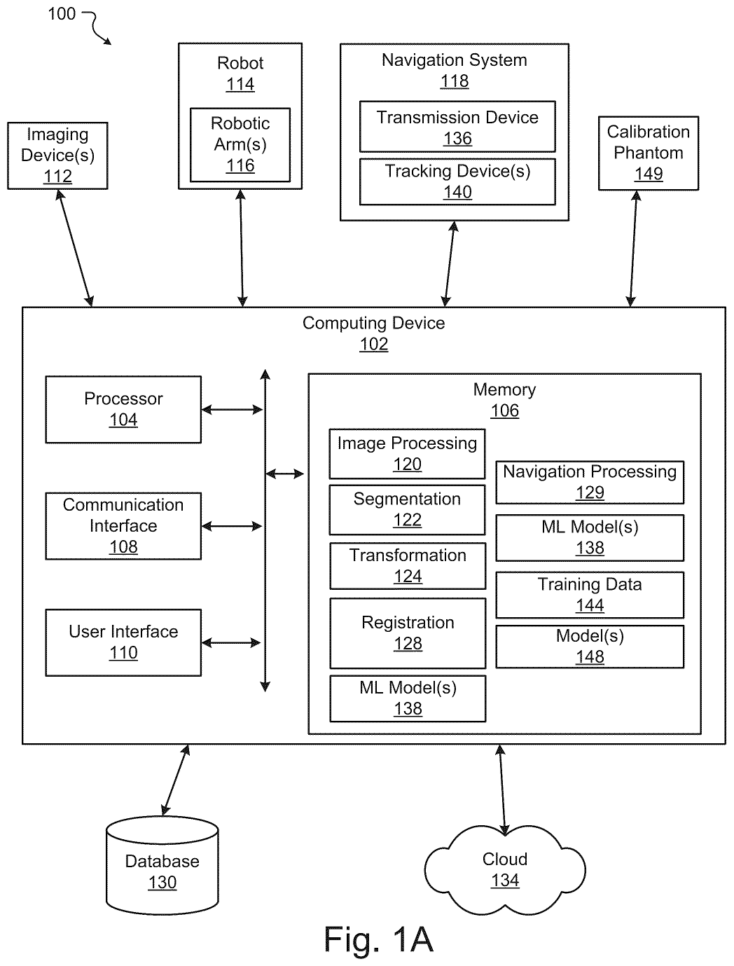

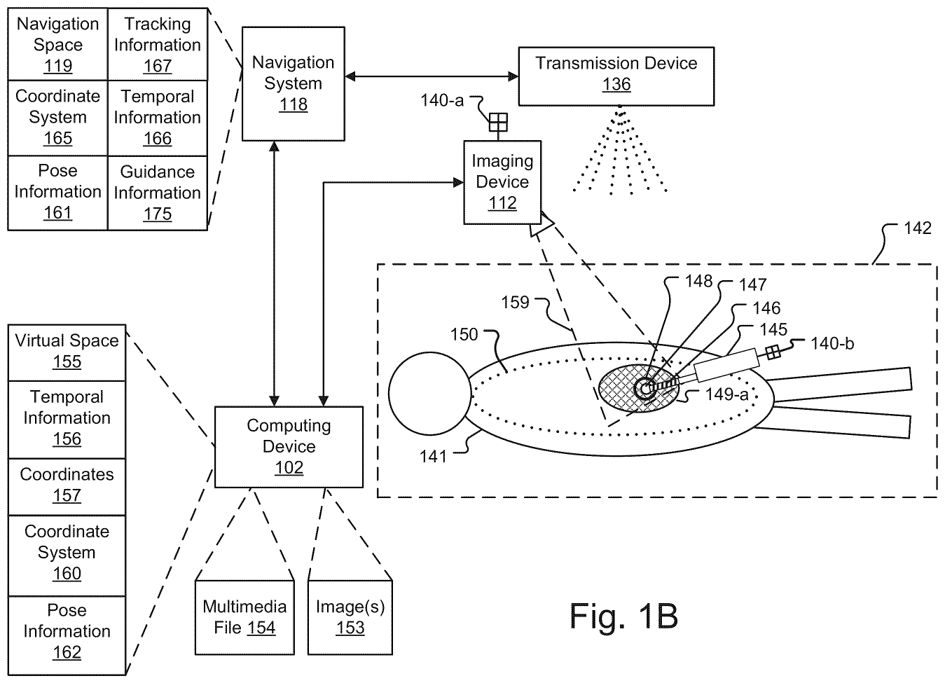

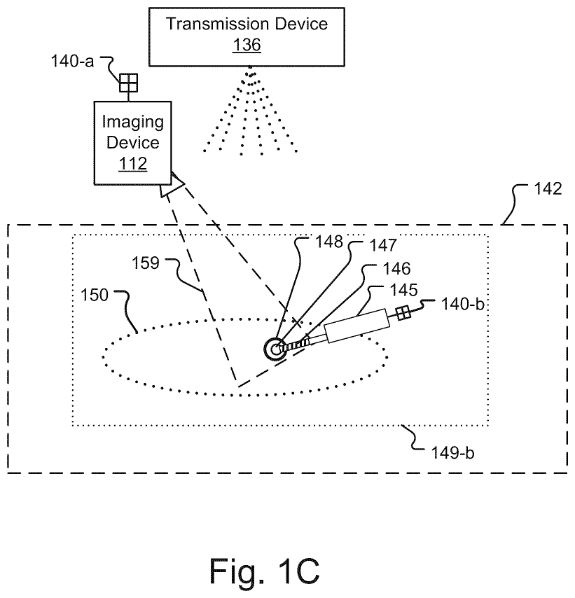

FIG. 1A illustrates an example of a system 100 that supports aspects of the present disclosure.

The system 100 includes a computing device 102, one or more imaging devices 112, a robot 114, a navigation system 118, a database 130, and/or a cloud network 134 (or other network). Systems according to other implementations of the present disclosure may include more or fewer components than the system 100. For example, the system 100 may omit and/or include additional instances of one or more components of the computing device 102, the imaging device(s) 112, the robot 114, navigation system 118, the database 130, and/or the cloud network 134. In an example, the system 100 may omit any instance of the computing device 102, the imaging device(s) 112, the robot 114, navigation system 118, the database 130, and/or the cloud network 134. The system 100 may support the implementation of one or more other aspects of one or more of the methods disclosed herein.

The computing device 102 includes a processor 104, a memory 106, a communication interface 108, and a user interface 110. Computing devices according to other implementations of the present disclosure may include more or fewer components than the computing device 102. The computing device 102 may be, for example, a control device including electronic circuitry associated with controlling any components of the system 100.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging devices 112, the robot 114, the navigation system 118, the database 130, and/or the cloud network 134.

The memory 106 may be or include RAM, DRAM, SDRAM, other solid-state memory, any memory described herein, or any other tangible, non-transitory memory for storing computer-readable data and/or instructions. The memory 106 may store information or data associated with completing, for example, any step of the process flow 500 described herein, or of any other methods. The memory 106 may store, for example, instructions and/or machine learning models that support one or more functions of the imaging devices 112, the robot 114, and the navigation system 118. For instance, the memory 106 may store content (e.g., instructions and/or machine learning models) that, when executed by the processor 104, enable image processing 120, segmentation 122, transformation 124, and/or registration 128. Such content, if provided as in instruction, may, in some implementations, be organized into one or more applications, modules, packages, layers, or engines.

Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging devices 112, the robot 114, the navigation system 118, the database 130, and/or the cloud network 134.

The computing device 102 may also include a communication interface 108. The communication interface 108 may be used for receiving data or other information from an external source (e.g., the imaging devices 112, the robot 114, the navigation system 118, the database 130, the cloud network 134, and/or any other system or component separate from the system 100), and/or for transmitting instructions, data (e.g., image data, tracking data, navigation data, calibration data, registration data, etc.), or other information to an external system or device (e.g., another computing device 102, the imaging devices 112, the robot 114, the navigation system 118, the database 130, the cloud network 134, and/or any other system or component not part of the system 100). The communication interface 108 may include one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some implementations, the communication interface 108 may support communication between the device 102 and one or more other processors 104 or computing devices 102, whether to reduce the time needed to accomplish a computing-intensive task or for any other reason.

The computing device 102 may also include one or more user interfaces 110. The user interface 110 may be or include a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some implementations, the user interface 110 may support user modification (e.g., by a surgeon, medical personnel, a patient, etc.) of instructions to be executed by the processor 104 according to one or more implementations of the present disclosure, and/or to user modification or adjustment of a setting of other information displayed on the user interface 110 or corresponding thereto.

In some implementations, the computing device 102 may utilize a user interface 110 that is housed separately from one or more remaining components of the computing device 102. In some implementations, the user interface 110 may be located proximate one or more other components of the computing device 102, while in other implementations, the user interface 110 may be located remotely from one or more other components of the computer device 102.

The imaging device 112 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may include data corresponding to an anatomical feature of a patient, or to a portion thereof. The image data may be or include a preoperative image, an intraoperative image, a postoperative image, or an image taken independently of any surgical procedure. In some implementations, a first imaging device 112 may be used to obtain first image data (e.g., a first image) at a first time, and a second imaging device 112 may be used to obtain second image data (e.g., a second image) at a second time after the first time.

The imaging device 112 may be capable of taking a 2D image or a 3D image to yield the image data. The imaging device 112 may be or include, for example, an ultrasound scanner (which may include, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may include, for example, a transmitter, a receiver, a processor, and one or more antennae), or any other imaging device 112 suitable for obtaining images of an anatomical feature of a patient. The imaging device 112 may be contained entirely within a single housing, or may include a transmitter/emitter and a receiver/detector that are in separate housings or are otherwise physically separated.

In some implementations, the imaging device 112 may include more than one imaging device 112. For example, a first imaging device may provide first image data and/or a first image, and a second imaging device may provide second image data and/or a second image. In still other implementations, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 114 may be any surgical robot or surgical robotic system. The robot 114 may be or include, for example, the Mazor X™ Stealth Edition robotic guidance system. The robot 114 may be configured to position the imaging device 112 at one or more precise position(s) and orientation(s), and/or to return the imaging device 112 to the same position(s) and orientation(s) at a later point in time. The robot 114 may additionally or alternatively be configured to manipulate a surgical tool (whether based on guidance from the navigation system 118 or not) to accomplish or to assist with a surgical task. In some implementations, the robot 114 may be configured to hold and/or manipulate an anatomical element during or in connection with a surgical procedure.

The robot 114 may include one or more robotic arms 116. In some implementations, the robotic arm 116 may include a first robotic arm and a second robotic arm, though the robot 114 may include more than two robotic arms. In some implementations, one or more of the robotic arms 116 may be used to hold and/or maneuver the imaging device 112. In implementations where the imaging device 112 includes two or more physically separate components (e.g., a transmitter and receiver), one robotic arm 116 may hold one such component, and another robotic arm 116 may hold another such component. Each robotic arm 116 may be positionable independently of the other robotic arm. The robotic arms 116 may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, an imaging device 112, surgical tool, or other object held by the robot 114 (or, more specifically, by the robotic arm 116) may be precisely positionable in one or more needed and specific positions and orientations.

The robotic arm(s) 116 may include one or more sensors that enable the processor 104 (or a processor of the robot 114) to determine a precise pose in space of the robotic arm (as well as any object or element held by or secured to the robotic arm).

In some implementations, reference markers (e.g., navigation markers) may be placed on the robot 114 (including, e.g., on the robotic arm 116), the imaging device 112, or any other object in the surgical space. The reference markers may be tracked by the navigation system 118, and the results of the tracking may be used by the robot 114 and/or by an operator of the system 100 or any component thereof. In some implementations, the navigation system 118 can be used to track other components (e.g., imaging device 112, surgical tools, instruments 145 (later described with reference to FIG. 1B), etc.) of the system and the system can operate without the use of the robot 114 (e.g., with the surgeon manually manipulating the imaging device 112 and/or one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers (e.g., tracking devices 140, etc.) or other objects within the operating room or other room in which some or all of the system 100 is located. The one or more cameras may be optical cameras, infrared cameras, or other cameras. In some implementations, the navigation system 118 may include one or more tracking devices 140 (e.g., electromagnetic sensors, acoustic sensors, etc.).

In some aspects, the navigation system 118 may include one or more of an optical tracking system, an acoustic tracking system, an electromagnetic tracking system, a radar tracking system, an inertial measurement unit (IMU) based tracking system, and a computer vision based tracking system. The navigation system 118 may include a corresponding transmission device 136 capable of transmitting signals associated with the tracking type. In some aspects, the navigation system 118 may be capable of computer vision based tracking of objects present in images captured by the imaging device(s) 112.

In various implementations, the navigation system 118 may be used to track a position and orientation (e.g., a pose) of the imaging device 112, the robot 114 and/or robotic arm 116, and/or one or more surgical tools (e.g., instrument 145, etc.) (or, more particularly, to track a pose of a navigated tracker attached, directly or indirectly, in fixed relation to the one or more of the foregoing). In some examples, the instrument 145 may be an electromagnetic pointer (or stylus). The navigation system 118 may include a display for displaying one or more images from an external source (e.g., the computing device 102, imaging device 112, or other source) or for displaying an image and/or video stream from the one or more cameras or other sensors of the navigation system 118.

In some implementations, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The processor 104 may utilize data stored in memory 106 as a neural network. The neural network may include a machine learning architecture. In some aspects, the neural network may be or include one or more classifiers. In some other aspects, the neural network may be or include any machine learning network such as, for example, a deep learning network, a convolutional neural network, a reconstructive neural network, a generative adversarial neural network, or any other neural network capable of accomplishing functions of the computing device 102 described herein. Some elements stored in memory 106 may be described as or referred to as instructions or instruction sets, and some functions of the computing device 102 may be implemented using machine learning techniques.

For example, the processor 104 may support machine learning model(s) 138 which may be trained and/or updated based on data (e.g., training data 144) provided or accessed by any of the computing device 102, the imaging device 112, the robot 114, the navigation system 118, the database 130, and/or the cloud network 134. The machine learning model(s) 138 may be built and updated based on the training data 144 (also referred to herein as training data and feedback).

The neural network and machine learning model(s) 138 may support AI/machine learning computer vision algorithms and object detection in association with automatically detecting, identifying, and tracking target objects (e.g., instruments, tools, etc.) in one or more images 153 or a multimedia file 154.

The database 130 may store information that correlates one coordinate system to another (e.g., one or more robotic coordinate systems, an ultrasound space coordinate system, a patient coordinate system, and/or a navigation coordinate system, etc.). The database 130 may additionally or alternatively store, for example, one or more surgical plans (including, for example, pose information about a target and/or image information about a patient's anatomy at and/or proximate the surgical site, for use by the robot 114, the ultrasound space coordinate system, the navigation system 118, and/or a user of the computing device 102 or of the system 100); one or more images 153 useful in connection with a surgery to be completed by or with the assistance of one or more other components of the system 100; and/or any other useful information.

The database 130 may be configured to provide any such information to the computing device 102 or to any other device of the system 100 or external to the system 100, whether directly or via the cloud network 134. In some implementations, the database 130 may include information associated with a calibration phantom 149 associated with a calibration procedure. In some implementations, the database 130 may be or include part of a hospital image storage system, such as a picture archiving and communication system (PACS), a health information system (HIS), and/or another system for collecting, storing, managing, and/or transmitting electronic medical records including image data.

In some aspects, the computing device 102 may communicate with a server(s) and/or a database (e.g., database 130) directly or indirectly over a communications network (e.g., the cloud network 134). The communications network may include any type of known communication medium or collection of communication media and may use any type of protocols to transport data between endpoints. The communications network may include wired communications technologies, wireless communications technologies, or any combination thereof.

Wired communications technologies may include, for example, Ethernet-based wired local area network (LAN) connections using physical transmission mediums (e.g., coaxial cable, copper cable/wire, fiber-optic cable, etc.). Wireless communications technologies may include, for example, cellular or cellular data connections and protocols (e.g., digital cellular, personal communications service (PCS), cellular digital packet data (CDPD), general packet radio service (GPRS), enhanced data rates for global system for mobile communications (GSM) evolution (EDGE), code division multiple access (CDMA), single-carrier radio transmission technology (1×RTT), evolution-data optimized (EVDO), high speed packet access (HSPA), universal mobile telecommunications service (UMTS), 3G, long term evolution (LTE), 4G, and/or 5G, etc.), Bluetooth®, Bluetooth® low energy, Wi-Fi, radio, satellite, infrared connections, and/or ZigBee® communication protocols.

The Internet is an example of the communications network that constitutes an Internet Protocol (IP) network consisting of multiple computers, computing networks, and other communication devices located in multiple locations, and components in the communications network (e.g., computers, computing networks, communication devices) may be connected through one or more telephone systems and other means. Other examples of the communications network may include, without limitation, a standard Plain Old Telephone System (POTS), an Integrated Services Digital Network (ISDN), the Public Switched Telephone Network (PSTN), a Local Arca Network (LAN), a Wide Area Network (WAN), a wireless LAN (WLAN), a Session Initiation Protocol (SIP) network, a Voice over Internet Protocol (VOIP) network, a cellular network, and any other type of packet-switched or circuit-switched network known in the art. In some cases, the communications network may include of any combination of networks or network types. In some aspects, the communications network may include any combination of communication mediums such as coaxial cable, copper cable/wire, fiber-optic cable, or antennas for communicating data (e.g., transmitting/receiving data).

The computing device 102 may be connected to the cloud network 134 via the communication interface 108, using a wired connection, a wireless connection, or both. In some implementations, the computing device 102 may communicate with the database 130 and/or an external device (e.g., a computing device) via the cloud network 134.

The system 100 or similar systems may be used, for example, to carry out one or more aspects of any of the process flow 500 described herein. The system 100 or similar systems may also be used for other purposes.

FIG. 1B illustrates an example of the system 100 that supports aspects of the present disclosure. Aspects of the example may be implemented by the computing device 102, imaging device(s) 112, robot 114 (e.g., a robotic system), and navigation system 118.

In some aspects, the navigation system 118 may provide navigation information based on an electromagnetic field generated by a transmission device 136. The navigation information may include tracking information 167 (also referred to herein as tracking data) as described herein. For example, the transmission device 136 may include an array of transmission coils capable of generating or forming the electromagnetic field in response to respective currents driven through the transmission coils. The navigation system 118 may include tracking devices 140 capable of sensing the electromagnetic field. Aspects of the navigation system 118 described herein may be implemented by navigation processing 129.

The system 100 may support tracking objects (e.g., an instrument 145, imaging device 112, etc.) in a trackable volume 150 using an electromagnetic field produced by the transmission device 136. For example, the transmission device 136 may include a transmitter antenna or transmitting coil array capable of producing the electromagnetic field. The system 100 may track the pose (e.g., position, coordinates, orientation, etc.) of the objects in the tracking volume 150 relative to a subject 141. In some aspects, the system 100 may display, via a user interface of the computing device 102, icons corresponding to any tracked objects. For example, the system 100 may superimpose such icons on and/or adjacent an image displayed on the user interface. The terms “tracking volume,” “trackable volume,” “navigation volume,” and “volume” may be used interchangeably herein.

In some aspects, the transmission device 136 may be an electromagnetic localizer that is operable to generate electromagnetic fields. The transmission device 136 may drive current through the transmission coils, thereby powering the coils to generate or form the electromagnetic field. As the current is driven through the coils, the electromagnetic field will extend away from the transmission coils and form a navigation domain (e.g., volume 150). The volume 150 may include any portion (e.g., the spine, one or more vertebrae, the brain, an anatomical element, or a portion thereof, etc.) of the subject 141 and/or any portion of a calibration phantom 149-a. The transmission coils may be powered through a controller device and/or power supply provided by the system 100.

The tracking devices 140 may include or be provided as sensors (also referred to herein as tracking sensors). The sensors may sense a selected portion or component of the electromagnetic field(s) generated by the transmission device 136. The navigation system 118 may support registration (e.g., through registration 128) of the volume 150 to a virtual space 155. The navigation system 118 may support superimposing an icon representing a tracked object (e.g., an instrument 145, a tracking device 140-b, a tracking device 146, etc.) on the image. The system 100 may support the delivery of tracking information associated with the tracking devices 140 and/or tracking device 146 to the navigation system 118. The tracking information may include, for example, data associated with magnetic fields sensed by the tracking devices 140.

The tracking devices 140 may communicate sensor information to the navigation system 118 for determining a position of the tracked portions relative to each other and/or for localizing an object (e.g., instrument 145, tracking device 146, etc.) relative to an image 153. The navigation system 118 and/or transmission device 136 may include a controller that supports operating and powering the generation of electromagnetic fields.

In the example of FIG. 1B, the system 100 may generate a navigation space 119 based on one or more tracking signals transmitted by the transmission device 136. The navigation space 119 may correspond to environment 142 or a portion thereof. For example, the navigation space 119 may correspond to a subject 141 (e.g., a patient) included in the environment 142 or an anatomical element (e.g., an organ, bone, tissue, etc.) of the subject 141. The environment 142 may be, for example, an operating room, an exam room, or the like. The tracking signals are not limited to electromagnetic tracking signals, and it is to be understood that the example aspects described with reference to FIG. 1B may be implemented using other types of tracking signals (e.g., optical tracking signals, acoustic tracking signals, etc.).

The system 100 may generate a virtual space 155 based on (e.g., in response to) signals transmitted by imaging device 112. The virtual space 155 may correspond to a field of view 159 of the imaging device 112. In an example, the system 100 may generate images 153 in response to signals transmitted by imaging device 112, and the images 153 may correspond to the field of view 159 of the imaging device 112. In the example of FIG. 1B, the imaging device 112 is an ultrasound probe transmitting ultrasound signals, and the images 153 may be ultrasound images.

The images 153 may be static images or video images. In some aspects, the images 153 may be stored as a multimedia file 154 that includes video (or video and sound). The imaging device 112 and the example signals transmitted by the imaging device 112 are not limited thereto, and it is to be understood that the example aspects described with reference to FIG. 1B may be implemented using other types of imaging devices 112 (e.g., X-ray, CT scanner, OCT scanner, etc.) and imaging systems described herein. The system 100 may support acquiring image data to generate or produce images (e.g., images 153, multimedia file 154, etc.) of the subject 141.

In an example, using the imaging device 112 and the transmission device 136, the system 100 may detect or track the calibration phantom 149-a and other objects (e.g., tracking devices 140, instruments 145, tracking device 146, etc.) included in the volume 150 and the virtual space 155. For example, at least a portion of the calibration phantom 149-a may be located in the volume 150 (as generated by the navigation system 118) and the virtual space 155 (as generated by the computing device 102). In the example of FIG. 1B, the calibration phantom 149-a is a tissue phantom, but is not limited thereto.

The system 100 may register and calibrate the imaging device 112 with respect to the navigation system 118. In the example of FIG. 1B, for an image 153 in which the calibration phantom 149-a is detected, the system 100 may identify a set of coordinates 157 in the virtual space 155 in response to an event in which the system 100 detects at least a portion of the instrument 145 in the image 153. In an example, the system 100 may detect that the portion of the instrument 145 is located in the calibration phantom 149-a and intersects a surface of the virtual space 155 at the set of coordinates 157. For example, the system 100 may detect that the portion of the instrument 145 intersects the surface at an angle perpendicular to the surface. In some examples, the portion of the instrument 145 may be a tracking device 146 (e.g., an electromagnetic antenna of the instrument 145).

In some example implementations, the virtual space 155 may be a 2D virtual space generated based on 2D images (e.g., ultrasound images, CT images, etc.) captured by the imaging device 112, and the surface may be a plane of the virtual space 155. In some other example implementations, the virtual space 155 may be a 3D virtual space (e.g., a volume), and the surface of the virtual space 155 may be a planar surface or non-planar surface.

According to example aspects of the present disclosure, the system 100 may calibrate the virtual space 155 to the navigation space 119 in response to the event in which the system 100 detects the instrument 145 (or at least a portion of the instrument 145) in the image 153. For example, in response to the event, the system 100 may calibrate a coordinate system 160 associated with the virtual space 155 with respect to a coordinate system 165 associated with the navigation space 119. In an example, the system 100 may calibrate the coordinate system 160 with respect to the coordinate system 165 based on the set of coordinates 157 and temporal information 156 associated with the event in which the instrument 145 (or portion of the instrument 145) intersects a surface (e.g., a plane, a volume, etc.) of the virtual space 155 at the set of coordinates 157. Further, for example, the system 100 may calibrate the coordinate system 160 with respect to the coordinate system 165 based on tracking information associated with the tracking device 146, the tracking device 140-b, and the tracking device 140-a.

For example, the instrument 145 may be registered to the navigation system 118 such that the navigation system 118 may track and determine pose information 161 of the instrument 145 based on tracking information 167 associated with the tracking device 146, the tracking device 140-a, and/or the tracking device 140-b and temporal information 166 corresponding to the tracking information 167. Further, for example, the navigation system 118 may track and determine pose information 161 of the imaging device 112 based on tracking information 167 associated with the tracking device 140-a and temporal information 166 corresponding to the tracking information 167. Accordingly, for example, the system 100 may calibrate the coordinate system 160 with respect to the coordinate system 165 based on the tracking information 167 (e.g., associated with the tracking device 146, the tracking device 140-b, and the tracking device 140-a), the temporal information 166 associated with the tracking information 167, the temporal information 156 associated with the event, and the coordinates 157 associated with the event.

The system 100 may detect, in an image 153 (or multimedia file 154), one or more landmarks corresponding to the instrument 145, a portion of the instrument 145, or the tracking device 146. The landmarks may correspond to distinctive features (e.g., a tip, a shape of the tip, etc.) of the instrument 145 or the tracking device 146. The system 100 may calibrate the coordinate system 160 with respect to the coordinate system 165 based on the one or more landmarks.

Aspects of the present disclosure support calibrating the virtual space 155 to the navigation space 119 (e.g., calibrating the coordinate system 160 associated with the virtual space 155 to the coordinate system 165 associated with the navigation space 119) in response to one or more criteria. For example, the system 100 may calibrate the virtual space 155 to the navigation space 119 in response to each occurrence of the event. In another example, the system 100 may calibrate the virtual space 155 to the navigation space 119 in response to each n^{th} occurrence of the event (e.g., each third occurrence of the event, each fifth occurrence, etc.). In some other examples, the system 100 may calibrate the virtual space 155 to the navigation space 119 in response to each n^{th} occurrence of the event within a temporal duration (e.g., each third occurrence of the event, in which the third occurrence is X seconds or less after a first occurrence of the event (where X is an integer value)). Accordingly, for example, aspects of the present disclosure support automatic registration during a surgical procedure, in which the registration is continuous or semi-continuous.

The system 100 may support calibrating the coordinate system 160 with respect to the coordinate system 165 without pausing a medical procedure (e.g., surgical procedure). In the example in which the calibration phantom 149-a is a tissue phantom inside the body of the subject 141, the system 100 may calibrate the coordinate system 160 with reference to the coordinate system 165 (e.g., recalibrate the registration between the virtual space 155 and the navigation space 119) in the background while medical personnel performs a medical procedure on the subject 141, without interrupting the medical procedure. That is, for example, the system 100 may calibrate the coordinate system 160 with reference to the coordinate system 165 during the medical procedure, without prompting the medical personnel to pause the medical procedure, such that the medical personnel may proceed with the medical procedure without waiting for calibration to be completed. In some aspects, the system 100 may calibrate the coordinate system 160 with reference to the coordinate system 165 without prompting the medical personnel to participate in a separate calibration operation.

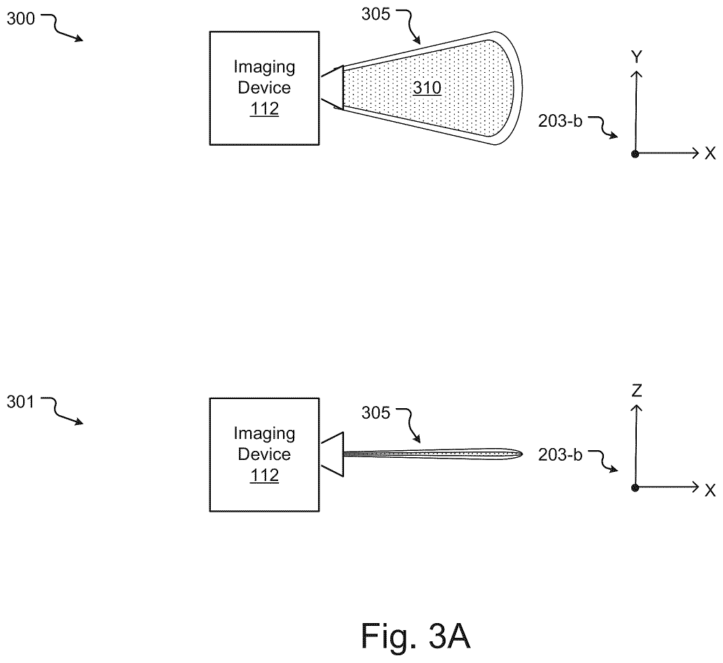

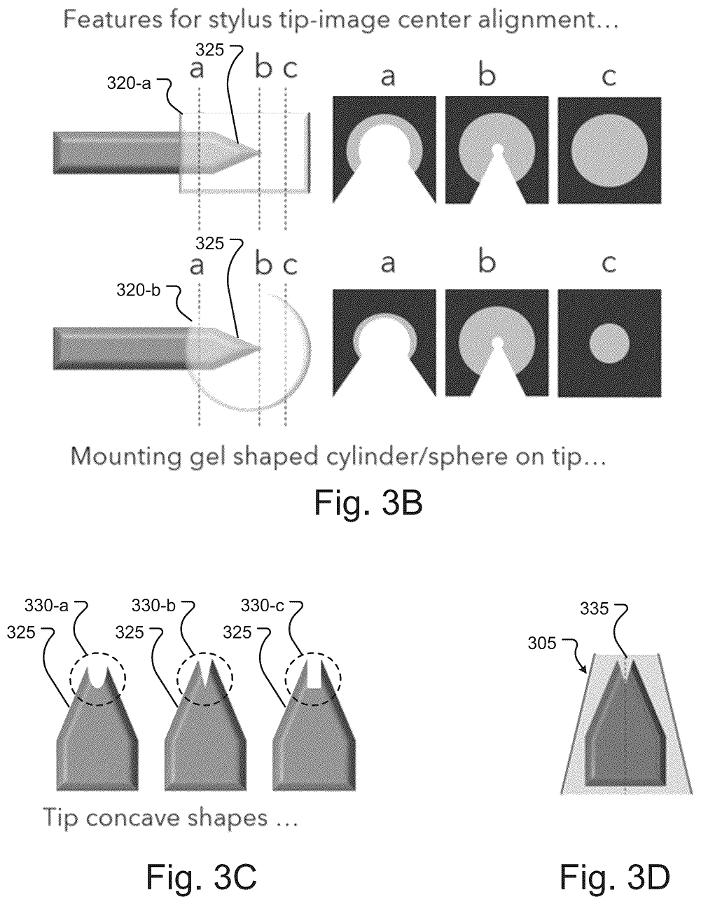

In some aspects, the system 100 and techniques described herein may support calibrating the coordinate system 160 with respect to the coordinate system 165 based on any of: properties (e.g., beam thickness, beam shape, signal frequency, etc.) of signals transmitted by the imaging device 112, pose information of the instrument 145 (or pose information of the tracking device 146) in association with an intersection between the instrument 145 (or tracking device 146) and the surface of the virtual space 155, and properties (e.g., shape, etc.) of the tracking device 146, example aspects of which are later described with reference to FIG. 3.

Aspects of calibrating the virtual space 155 to the navigation space 119 (e.g., calibrating the coordinate system 160 associated with the virtual space 155 to the coordinate system 165 associated with the navigation space 119) include verifying a registration accuracy between the coordinate system 160 and the coordinate system 165. For example, in response to an occurrence of the event as described herein, the system 100 may calculate a registration accuracy between the coordinate system 160 and the coordinate system 165 and compare the registration accuracy to a target accuracy value. In an example, in response to a comparison result in which the registration accuracy is less than the target accuracy value, the system 100 may perform one or more operations described herein in association with recalibrating the virtual space 155 to the navigation space 119. For example, the system 100 may autonomously recalibrate the coordinate system 160 to the coordinate system 165. In another example, the system 100 may generate and output a notification including user guidance information 175 (e.g., tutorials, user prompts, corrective actions, real-time actional feedback, etc.) regarding how to move or position a device (e.g., the imaging device 112, the instrument 145, etc.) in association with the calibration process. In some examples, the notification may include a visual notification, an audible notification, a haptic notification, or a combination thereof.

Other additional and/or alternative aspects of calibrating the virtual space 155 to the navigation space 119 include automatically detecting distortion in the navigated volume 150 due to discrepancies between navigation data (e.g., tracking information 167 provided by navigation system 118) and imaging data (e.g., images 153, multimedia file 154, etc.). In an example, the discrepancies may be between pose information 161 of a tracked object (e.g., tracking device 140-b, instrument 145, tracking device 146, etc.) as indicated by the navigation system 118 and pose information 162 of the tracked object as determined by the computing device 102 from the imaging data.

In an example implementation, in response to an occurrence of the event as described herein, the system 100 may calculate the discrepancy and compare the discrepancy to a target discrepancy threshold value. In an example, in response to a comparison result in which the discrepancy is greater than the discrepancy threshold value, the system 100 may perform one or more operations described herein (e.g., autonomous recalibration, outputting a notification including user guidance information 175, etc.) in association with recalibrating the virtual space 155 to the navigation space 119. In some aspects, the system 100 may calibrate the navigation data to the imaging data (e.g., calibrate the navigation space 119 to the virtual space 155) while compensating for the discrepancies.

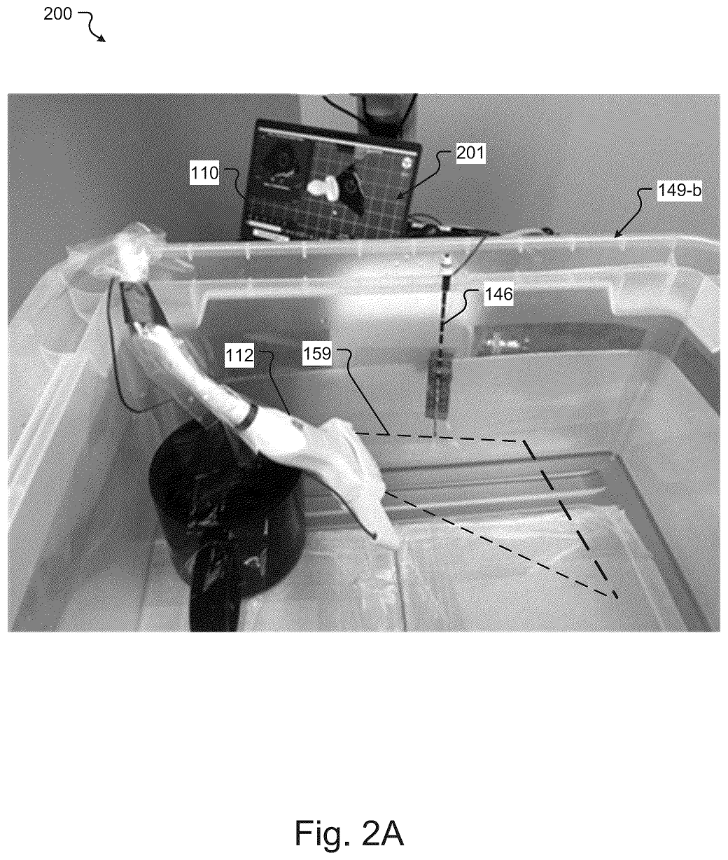

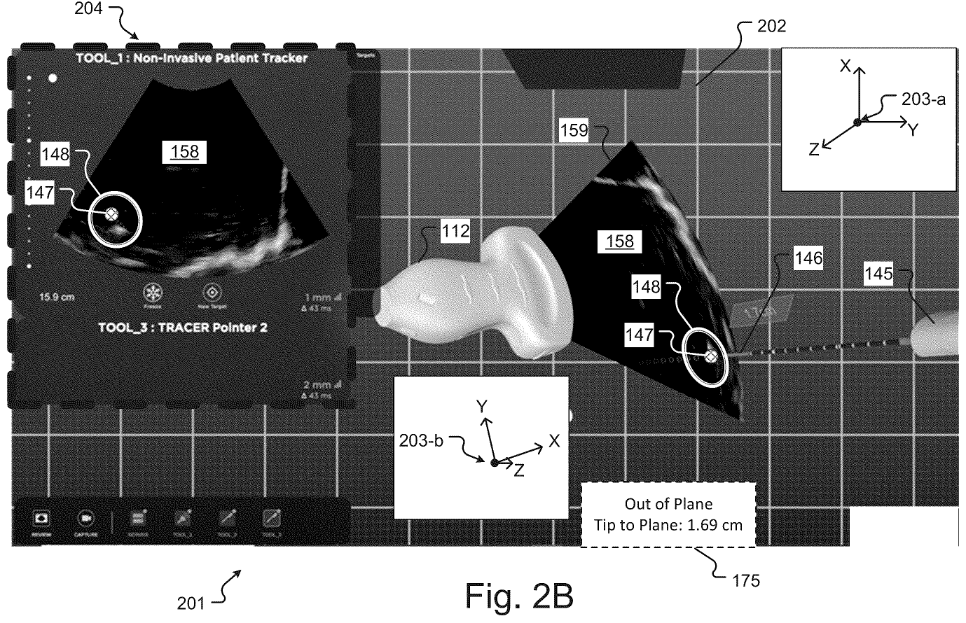

In an example, the system 100 may identify that tracking device 146 (e.g., electromagnetic antenna of an instrument 145) is intersecting the plane of an ultrasound imaging field 158 at a point 147 of intersection inside a circle 148, example aspects of which will later be described with reference to FIG. 2B.

The techniques described herein may provide continuous automatic registration, continuous semi-automatic registration, or a combination thereof, and the registration techniques may be implemented during a medical procedure.

FIG. 1C illustrates an example of the system 100 that supports aspects of the present disclosure. Aspects of the example in FIG. 1C include like aspects described with reference to FIG. 1B. Referring to the example of FIG. 1C, the calibration phantom 149-b may be an ultrasound transmitting volume (e.g., a water bath) implemented using an empty container (e.g., an empty box including an electromagnetic friendly material). In some aspects, the calibration phantom 149-b may include ultrasound conductive material.

For example, the container may be formed of low magnetic or non-magnetic materials so as to minimize distortion to electromagnetic fields. In an example, the container may be formed of a material having a magnetic permeability of about 1.0 to about 1.1 (relative), and the material may have a relatively low electrical conductivity (e.g., an electrical conductivity less than a threshold value). In some examples, the material may be a stainless steel alloyed with different metallic elements associated with obtaining specific properties (e.g., temperature and corrosion resistance, fracture tolerance, etc.). Non-limiting examples of the material include Nickel/Chromium alloys (e.g., Series 300 alloys, type 304 stainless steel (annealed condition only), type 316 stainless steel), Cobalt/Chromium alloys (e.g., L605, MP35N), and Titanium alloys (e.g., Ti6Al4V), plastics, and wood.

In an example, one or more surfaces of the container may include cross wires with patterns, and the container is full of water.

In some examples, the calibration phantom 149-b may be integrated into the structure of a patient tracker. In some other aspects, the calibration phantom 149-b may be included in the environment 142 as a standalone structure that is separate from an operating table associated with the subject 141. Example aspects of the water bath are later described with reference to FIG. 2A.

According to example aspects of the present disclosure, the system 100 may support calibrating the virtual space 155 to the navigation space 119 using the calibration phantom 149-b and the techniques as described herein, in which the calibration phantom 149-b is substituted for the calibration phantom 149-a described with reference to FIG. 1B.

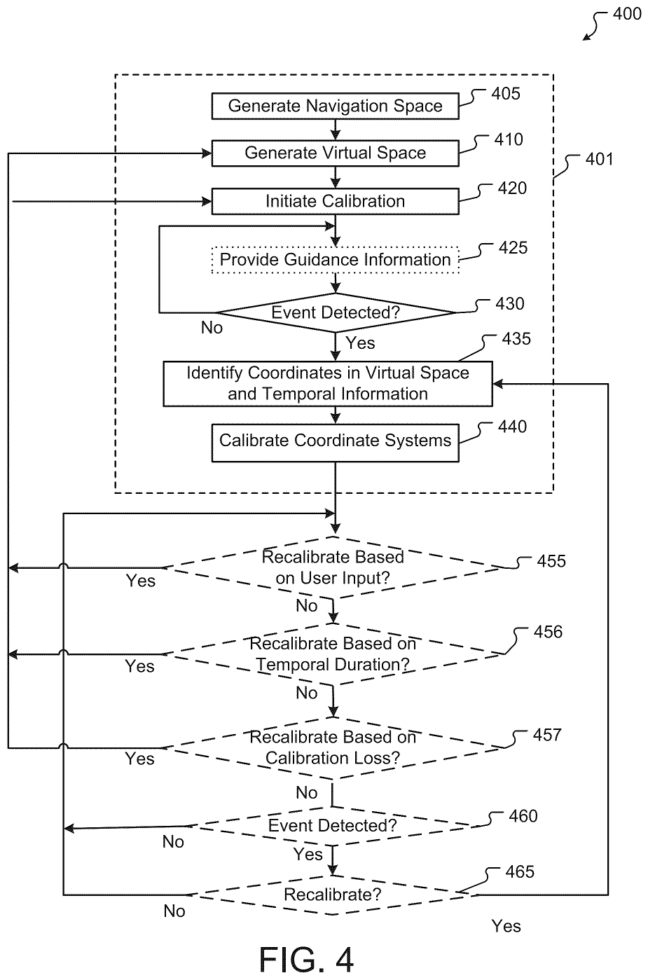

In some example implementations, the system 100 may support calibrating the virtual space 155 to the navigation space 119 using both the calibration phantom 149-a and the calibration phantom 149-b. For example, the system 100 may support calibration outside the subject 141 using the calibration phantom 149-b (e.g., water bath) and further calibration (e.g., recalibration, calibration adjustment, etc.) using the calibration phantom 149-a (e.g., tissue of the subject 141), and the combination may provide an increase in accuracy compared to other calibration techniques. An example implementation of using both the calibration phantom 149-a and the calibration phantom 149-b in association with a calibration process is later described with reference to FIG. 4.

FIG. 2A illustrates an example implementation 200 of the system 100. Referring to FIG. 2A, an imaging device 112 (e.g., an electromagnetic tracked ultrasound probe) may be inserted in calibration phantom 149-b (e.g., a water bath). A transmission device (e.g., an electromagnetic emitter) may be positioned inside or within a threshold distance of the calibration phantom 149-b. In the example view of FIG. 2A, the transmission device is positioned outside of (e.g., behind) the calibration phantom 149-b.

A tracking device 146 may be positioned in an ultrasound imaging field 158 (later illustrated at FIG. 2B) associated with the imaging device 112 at multiple points by moving the tracking device 146 through the ultrasound imaging field. In the example implementation 200, the ultrasound imaging field 158 corresponds to the field of view 159 of the imaging device 112. A navigation space 119 corresponding to the calibration phantom 149-b may be generated by the navigation system 118 as described herein, and the system 100 may display a virtual representation 201 of the navigation space 119 and the virtual space 155 via, for example, a user interface 110. The tracking device 146 may be referred to as a navigated instrument. In the example of FIG. 2A, the tracking device 146 is a navigation pointer (e.g., stylus).

Example aspects of the virtual representation 201 of the navigation space 119 and the virtual space 155 are later described with reference to FIG. 2B.

FIG. 2B illustrates an example of the virtual representation 201 of the navigation space 119 and the virtual space 155.

The virtual representation 201 may include a multi-dimensional representation corresponding to the volume of the calibration phantom 149-b. The virtual representation 201 may include the imaging device 112, the instrument 145, the instrument 145 (or portions of the instrument 145), and/or tracking device 146. In some aspects, the virtual representation 201 may include a pattern (e.g., represented by lines 202 and/or dots) that correspond to the patterns described with reference to the container used for implementing the calibration phantom 149-b.

Features of the virtual representation 201 may be described in conjunction with a coordinate systems 203 (e.g., coordinate system 203-a, coordinate system 203-b). Each coordinate system 203, as shown in FIG. 2B, includes three-dimensions including an X-axis, a Y-axis, and a Z-axis. Additionally or alternatively, coordinate system 203-a may be used to define surfaces, planes, or volume of the calibration phantom 149-b and/or the navigation space 119. The virtual representation 201 may include coordinate system 203-b that corresponds to the imaging device 112.

The planes of each coordinate system 203 (e.g., coordinate system 203-a, coordinate system 203-b) may be disposed orthogonal, or at 90 degrees, to one another. While the origin of a coordinate system 203 may be placed at any point on or near the components of the navigation system 118, for the purposes of description, the axes of the coordinate system 203 are always disposed along the same directions from figure to figure, whether the coordinate system 203 is shown or not. In some examples, reference may be made to dimensions, angles, directions, relative positions, and/or movements associated with one or more components of the imaging device 112 and/or the navigation system 118 with respect to a coordinate system 203.

Referring to the virtual representation 201 illustrated in FIG. 2B, a tracking device 146 (e.g., electromagnetic antenna of an instrument 145) is intersecting the plane of the ultrasound imaging field 158 at a point 147 of intersection inside a circle 148. In an example, the point 147 may be an echogenic dot. In some aspects, the virtual representation 201 may include a window 204 displaying the imaging field 158 and information (e.g., point 147, circle 148, etc.) associated with the imaging field 158. The system 100 may display guidance information 175 indicating pose information of the tracking device 146 with respect to the ultrasound imaging field 158. In some aspects, the user guidance information 175 may include an indication whether the tracking device 146 is outside the plane of the ultrasound imaging field 158 (e.g., ‘Out of Plane’) or intersecting the plane of the ultrasound imaging field 158 (e.g., at a point 147). In some aspects, the guidance information 175 may include distance information (e.g., ‘Tip to Plane: 1.69 cm’) of the tracking device 146 with respect to the ultrasound imaging field 158.

It is to be understood that the aspects described herein with reference to the plane of the ultrasound imaging field 158 support implementations applied to any surface of a virtual space 155 (e.g., a plane of the virtual space 155 for cases in which the virtual space 155 is a 2D virtual space, a planar surface or non-planar surface of the virtual space 155 for cases in which the virtual space 155 is a 3D virtual space, etc.). It is to be understood that the aspects described herein may be applied to an electromagnetic antenna, a navigation stylus, a pointer, or any navigated tools having a geometry and location that is defined, known, and trusted by the system 100.

The system 100 may record all navigation data (e.g., electromagnetic data), imaging data (e.g., ultrasound data), and corresponding temporal information (e.g., temporal information 156, temporal information 166) in a multimedia file 154. In an example, the multimedia file 154 may be a movie file, and the system 100 may record timestamps corresponding to the navigation data and the imaging data. Based on the navigation data, the imaging data, and the temporal information, the system 100 may identify when the tip of the tracking device 146 enters the ultrasound field of view 159 and intersects the plane of the ultrasound imaging field 158. Based on the identification of when the tip of the tracking device 146 enters the ultrasound field of view 159 and intersects the plane of the ultrasound imaging field 158, the system 100 may verify the calibration of the imaging device 112 with the electromagnetic navigation of the navigation system 118.

FIG. 3A illustrates example views 300 and 301 of an ultrasound beam 305 transmitted by the imaging device 112 when viewed from different perspective views. Referring to the example view 300, the ultrasound beam 305 is relatively thick (or wide) with respect to the Y-axis. In contrast, referring to the example view 301, the ultrasound beam 305 is relatively narrow with respect to the Z-axis. In the example views 300 and 301 of the ultrasound beam 305, thickness varies in depth, and the shape and focal point of the ultrasound beam 305 may be based on parameters (e.g., power, frequency, etc.) of the ultrasound beam 305.

According to example aspects of the present disclosure, referring to example view 300, the system 100 may calibrate the virtual space 155 to the navigation space 119 based on instances in which the instrument 145 (or tracking device 146) intersects a cross-sectional area 310 (e.g., an area in the XY plane) of the ultrasound beam 305 in a direction along the Z axis. That is, for example, the system 100 may perform the calibration for instances in which the instrument 145 or tracking device 146 intersects a portion of the cross-sectional area 310 (e.g., the length of the instrument 145 or tracking device 146 is perpendicular (or near perpendicular) the cross-sectional area 310), while factoring in values of parameters (e.g., thickness, depth, shape, focal point, etc.) of the ultrasound beam 305.

In another example, referring to example view 301, the system 100 may calibrate the virtual space 155 to the navigation space 119 for instances in which the instrument 145 (or tracking device 146) intersects the ultrasound beam 305 in the direction along the Z axis. That is, for example, the system 100 may perform the calibration for instances in which the instrument 145 or tracking device 146 intersects the ultrasound beam 305, while incorporating values of parameters (e.g., thickness, depth, shape, focal point, etc.) of the ultrasound beam 305.

FIG. 3B illustrates example elements 320 (e.g., element 320-a, element 320-b) that may be implemented at instrument 145 (e.g., at a tip 325 of the instrument 145, at a tip 325 of tracking device 146, etc.) in association with calibrating the virtual space 155 to the navigation space 119. In an example implementation, element 320-a may be a gel that is cylinder shaped. In another example implementation, element 320-b may be a gel that is sphere shaped.

FIG. 3C illustrates example shapes 330 (e.g., shapes 330-a through 330-c) that may be implemented at a tip 325 of the instrument 145, at a tip 325 of tracking device 146, or the like. Aspects of the present disclosure may include implementing any of the shapes 330, and the shapes 330 may support tip-image center alignment. That is, for example, each shape 330 may be symmetrical with respect to a center of the shape 330, which may support alignment of the center of the shape 330 and a center 335 of an ultrasound beam 305 emitted by the imaging device 112, an example of which is illustrated at FIG. 3D.

FIG. 4 illustrates an example of a process flow 400 in accordance with aspects of the present disclosure. In some examples, process flow 400 may implement aspects of a computing device 102, an imaging device 112, a robot 114, and a navigation system 118 described with reference to FIGS. 1 through 3.

In the following description of the process flow 400, the operations may be performed in a different order than the order shown, or the operations may be performed in different orders or at different times. Certain operations may also be left out of the process flow 400, or other operations may be added to the process flow 400.

It is to be understood that any of the operations of process flow 400 may be performed by any device (e.g., a computing device 102, an imaging device 112, a robot 114, navigation system 118, etc.) of the system 100 described herein.

At 405, the system 100 may generate a navigation space 119 as described herein. In the example of the process flow 400.

At 410, the system 100 may generate a virtual space 155 based on images captured by the imaging device 112 as described herein. For example, the system 100 may generate the virtual space 155 based on images representing the inside of calibration phantom 149-b (e.g., water bath).

At 420, the system 100 may initiate a calibration process 401 in accordance with aspects of the present disclosure. For example, at 420, the system 100 may initiate calibration of the coordinate system 160 associated with the virtual space 155 (and imaging device 112) with respect to the coordinate system 165 associated with the navigation space 119 (and navigation system 118).

At 425, the system 100 may provide user guidance information 175 (e.g., tutorials, user prompts, corrective actions, real-time actional feedback, etc.) described herein regarding how to move or position a device (e.g., the imaging device 112, the instrument 145, etc.) in association with the calibration process. The terms “guidance information” and “calibration guidance information” may be used interchangeably herein. Aspects of the present disclosure support implementations with or without providing user guidance information 175.

At 430 (Event Detected?= ‘Yes’), the system 100 may determine, from an image 153 (or multimedia file 154), whether an event has occurred in which the instrument 145 (or portion of the instrument 145) intersects a surface (e.g., a plane, a volume, etc.) of the virtual space 155. In another example, at 430 (Event Detected?= ‘No’), the system 100 may analyze subsequent images 153 (or multimedia files 154) until the system 100 detects an event in which the instrument 145 (or portion of the instrument 145) intersects a surface (e.g., a plane, a volume, etc.) of the virtual space 155. In some aspects, the system 100 may return to 425 and provide additional user guidance information 175 that prompts a user to position or orient the imaging device 112 and/or the instrument 145 to trigger such an event.

At 435, the system 100 may identify, from the image 153 (or multimedia file 154) the set of coordinates 157 at which the instrument 145 (or portion of the instrument 145) intersects a surface (e.g., a plane, a volume, etc.) of the virtual space 155. In some aspects, at 435, the system 100 may identify the temporal information 156 associated with when the instrument 145 intersected the surface of the virtual space 155. In some cases, the system 100 may identify pose information 162 (in the virtual space 155) of the instrument 145 that corresponds to the temporal information 156.

At 440, the system 100 may calibrate the coordinate system 160 with respect to the coordinate system 165 based on the set of coordinates 157 and the temporal information 156 as described herein. In an example, the system 100 may calibrate the coordinate system 160 with respect to the coordinate system 165 based on the set of coordinates 157, the temporal information 156, the pose information 161 (of the instrument 145 in the virtual space 155) corresponding to the temporal information 156, and pose information 162 (of the instrument 145 in the navigation space 119) corresponding to the temporal information 156.

At 455 through 457, the system 100 may determine whether to repeat the calibration process 401.

For example, at 455, the system 100 may determine whether a user input requesting recalibration has been received. In response to receiving a user input requesting recalibration, the system 100 may determine that recalibration is to be performed (e.g., Recalibrate Based on User Input?= ‘Yes’).

In another example, at 456, the system 100 may determine whether a temporal duration (e.g., recalibration every X hours, every day, etc.) associated with performing recalibration has elapsed. In response to identifying that the temporal duration has elapsed, the system 100 may determine that recalibration is to be performed (e.g., Recalibrate Based on Temporal Duration?= ‘Yes’).

In some other examples, at 457, the system 100 may detect for any losses in calibration between the imaging device 112 and the navigation system 118 (e.g., the navigation system 118 is unable to track the imaging device 112) has occurred. In response to detecting a loss in calibration between the imaging device 112 and the navigation system 118 (e.g., the navigation system 118 is unable to track the imaging device 112), the system 100 may determine that recalibration is to be performed (e.g., Recalibrate Based on Calibration Loss?= ‘Yes’).

According to example aspects of the present disclosure, based on decisions by the system 100 at any of 455 through 457, the system 100 may repeat the calibration process 401, beginning at any operation (e.g., generating the virtual space 155 at 410, initiating calibration at 420, etc.) of the calibration process 401. In an example, in response to a ‘Yes’ decision at any of 455 through 457, the system 100 may return to 410 and generate the virtual space 155, but while navigating the calibration phantom 149-a (e.g., tissue phantom) with the imaging device 112. For example, in repeating the calibration process 401, the system 100 may regenerate the virtual space 155 based on images captured by the imaging device 112 as described herein, but the images may be associated with or include calibration phantom 149-a (e.g., tissue phantom).

In an example implementation, after repeating the calibration process 401, the system 100 may again return to 410 in response to a ‘Yes’ decision at any of 455 through 457 and generate the virtual space 155, while imaging the calibration phantom 149-a (e.g., tissue phantom) with the imaging device 112.

In an alternative or additional example, in response to a ‘No” decision at any of 455 through 457, the system 100 may continue to provide navigation information (e.g., tracking information 167, etc.).

While providing imaging information (e.g., images 153, etc.) and navigation information (e.g., tracking information 167, etc.), the system 100 may monitor for one or more events in which the instrument 145 (or portion of the instrument 145) intersects a surface (e.g., a plane, a volume, etc.) of the virtual space 155 as described herein. In an example, at 460, the system 100 may detect an event in which the instrument 145 (or portion of the instrument 145) intersects a surface of the virtual space 155 (Event Detected?= ‘Yes’).

At 465, the system 100 may determine, from the event detected at 460, whether recalibration is to be performed.

In an example implementation, the system 100 may detect the amount of distortion in the navigated volume 150 (e.g., discrepancies between navigation data associated with the instrument 145 and imaging data associated with the instrument 145) based on the event detected at 460. Based on the amount of distortion detected by the system 100, the system 100 may return to 435 (e.g., for recalibration) or refrain from returning to 435 (e.g., abstain from performing recalibration).

For example, the system 100 may determine that the amount of distortion is greater than a threshold distortion value, and at 465 (e.g., Recalibrate?= ‘Yes’), the system 100 may return to 435 and repeat the calibration as described with reference to 440. In another example, the system 100 may determine that the amount of distortion is less than the threshold distortion value, and at 465 (e.g., Recalibrate?= ‘No’), and the system 100 may continue to provide imaging information and/or navigation information (e.g., tracking information 167, etc.) while monitoring for any of the events described with reference to 455 through 460.

As supported by aspects of the present disclosure, the system 100 may determine (at 465) whether to perform recalibration, at any occurrence of an event detected at 460. For example, the system 100 may perform recalibration at each occurrence of an event detected at 460, at each n^{th} occurrence of the event, or at each n^{th} occurrence of the event within a temporal duration.

As illustrated and described herein, the example aspects of the process flow 400 described herein support automatic and continuous (or semi-continuous) recalibration by the system 100.

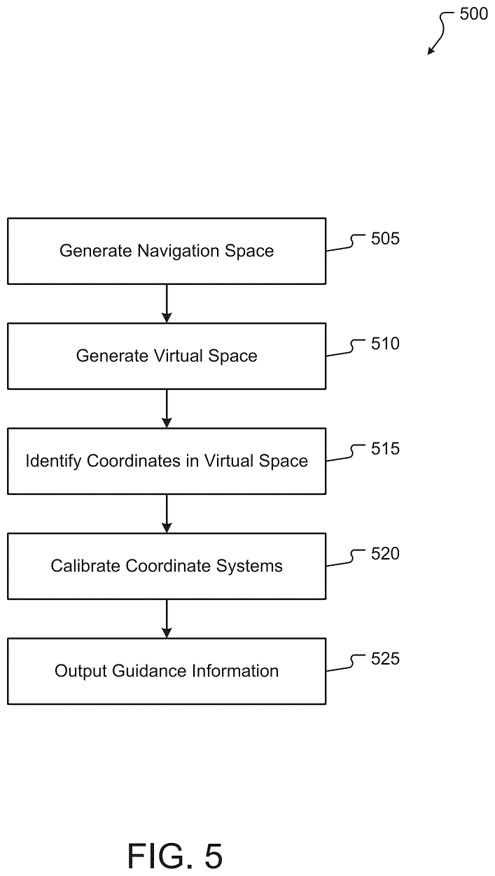

FIG. 5 illustrates an example of a process flow 500 in accordance with aspects of the present disclosure. In some examples, process flow 500 may implement aspects of a computing device 102, an imaging device 112, a robot 114, and a navigation system 118 described with reference to FIGS. 1 through 3.

In the following description of the process flow 500, the operations may be performed in a different order than the order shown, or the operations may be performed in different orders or at different times. Certain operations may also be left out of the process flow 500, or other operations may be added to the process flow 500.

It is to be understood that any of the operations of process flow 500 may be performed by any device (e.g., a computing device 102, an imaging device 112, a robot 114, navigation system 118, etc.) of the system 100 described herein.

At 505, the process flow 500 may include generating a navigation space based on one or more tracking signals.

At 510, the process flow 500 may include generating a virtual space including at least a portion of a calibration phantom based on one or more images, wherein the one or more images are generated in response to transmitting one or more imaging signals.

In some aspects, the calibration phantom includes an ultrasound conductive material. For example, the calibration phantom may include an ultrasound transmitting volume (e.g., water bath). In some aspects, the calibration phantom includes a tissue phantom included in a body of a subject, wherein the tissue phantom and the subject are associated with a surgical procedure.

In some aspects, the virtual space corresponds to a field of view of the imaging device.

At 515, the process flow 500 may include identifying a set of coordinates in the virtual space in response to an event in which at least a portion of a tracked device in the calibration phantom is detected in the one or more images.

In some aspects, the set of coordinates are identified in the virtual space in response to at least the portion of the tracked device intersecting a plane of the virtual space at the set of coordinates.

In some aspects, the navigation space and the tracked device are associated with at least one of: an optical tracking system, an acoustic tracking system, an electromagnetic tracking system, a magnetic tracking system, a radar tracking system, an inertial measurement unit (IMU) based tracking system, and a computer vision based tracking system.

At 520, the process flow 500 may include calibrating a first coordinate system associated with the virtual space with respect to a second coordinate system associated with the navigation space in response to the event, wherein calibrating the first coordinate system with respect to the second coordinate system is based on the set of coordinates and temporal information associated with the event.

In some aspects, calibrating the first coordinate system with respect to the second coordinate system is in response to one or more occurrences of the event. In some aspects, calibrating the first coordinate system with respect to the second coordinate system is absent pausing the surgical procedure.

In some aspects, calibrating the first coordinate system with respect to the second coordinate system is based on: beam thickness (e.g., ultrasound beam thickness), beam shape (e.g., ultrasound beam shape), or both of the one or more signals transmitted by the imaging device; pose information of the portion of the tracked device in association with an intersection between the portion of the tracked device and a plane of the virtual space; and one or more properties of the portion of the tracked device.

In some aspects, the tracked device is comprised in at least a portion of an instrument, and the process flow 500 may include detecting, in the one or more images, one or more landmarks corresponding to at least a portion of the tracked device, wherein calibrating the first coordinate system with respect to the second coordinate system is based on the one or more landmarks.

In some aspects, calibrating the first coordinate system with respect to the second coordinate system includes verifying a registration accuracy between the first coordinate system and the second coordinate system.

At 525, the process flow 500 may include outputting guidance information associated with positioning the imaging device, the tracked device, or both in association with calibrating the first coordinate system with respect to the second coordinate system.

In some aspects, the process flow 500 may include detecting one or more discrepancies between first tracking data corresponding to the tracked device in association with the navigation space and second tracking data corresponding to the tracked device in association with the virtual space. In some aspects, the process flow 500 may include generating a notification associated with the one or more discrepancies, performing one or more operations associated with compensating for the one or more discrepancies, or both.

The process flow 500 (and/or one or more operations thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the process flow 500. The at least one processor may perform operations of the process flow 500 by executing elements stored in a memory such as the memory 106. The elements stored in memory and executed by the processor may cause the processor to execute one or more operations of a function as shown in the process flow 500. One or more portions of the process flow 500 may be performed by the processor executing any of the contents of memory, such as image processing 120, a segmentation 122, a transformation 124, and/or a registration 128.

As noted above, the present disclosure encompasses methods with fewer than all of the steps identified herein (and the corresponding description of respective process flows), as well as methods that include additional steps beyond those identified in the figures and process flows described herein). The present disclosure also encompasses methods that include one or more steps from one method described herein, and one or more steps from another method described herein. Any correlation described herein may be or include a registration or any other correlation.

The foregoing is not intended to limit the disclosure to the form or forms disclosed herein. In the foregoing Detailed Description, for example, various features of the disclosure are grouped together in one or more aspects, implementations, and/or configurations for the purpose of streamlining the disclosure. The features of the aspects, implementations, and/or configurations of the disclosure may be combined in alternate aspects, implementations, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, implementation, and/or configuration. Thus, the following claims are hereby incorporated into this Detailed Description, with each claim standing on its own as a separate preferred implementation of the disclosure.

Moreover, though the foregoing has included description of one or more aspects, implementations, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, implementations, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

Example aspects of the present disclosure include:

A system including: a processor; and a memory storing instructions that, when executed by the processor, cause the processor to: generate a navigation space based on one or more tracking signals; generate a virtual space including at least a portion of a calibration phantom based on one or more images, wherein the one or more images are generated in response to one or more signals transmitted by an imaging device; identify a set of coordinates in the virtual space in response to an event in which at least a portion of a tracked device in the calibration phantom is detected in the one or more images; and calibrate a first coordinate system associated with the virtual space with respect to a second coordinate system associated with the navigation space in response to the event, wherein calibrating the first coordinate system with respect to the second coordinate system is based on the set of coordinates and temporal information associated with the event.

Any of the aspects herein, wherein the set of coordinates are identified in the virtual space in response to at least the portion of the tracked device intersecting a surface of the virtual space at the set of coordinates.

Any of the aspects herein, wherein: calibrating the first coordinate system with respect to the second coordinate system is in response to one or more occurrences of the event; and calibrating the first coordinate system with respect to the second coordinate system is absent pausing a surgical procedure.

Any of the aspects herein, wherein calibrating the first coordinate system with respect to the second coordinate system is based on: beam thickness, beam shape, or both of the one or more signals transmitted by the imaging device; pose information of the portion of the tracked device in association with an intersection between the portion of the tracked device and a plane of the virtual space; and one or more properties of the portion of the tracked device.

Any of the aspects herein, wherein the calibration phantom includes: a water bath; or a tissue phantom included in a body of a subject, wherein the tissue phantom and the subject are associated with a surgical procedure.

Any of the aspects herein, wherein the instructions are further executable by the processor to: output guidance information associated with positioning the imaging device, the tracked device, or both in association with calibrating the first coordinate system with respect to the second coordinate system.

Any of the aspects herein, wherein the tracked device is included in at least a portion of an instrument, and the instructions are further executable by the processor to: detect, in the one or more images, one or more landmarks corresponding to at least a portion of the tracked device, wherein calibrating the first coordinate system with respect to the second coordinate system is based on the one or more landmarks.

Any of the aspects herein, wherein calibrating the first coordinate system with respect to the second coordinate system includes verifying a registration accuracy between the first coordinate system and the second coordinate system.

Any of the aspects herein, wherein the instructions are further executable by the processor to: detect one or more discrepancies between first tracking data corresponding to the tracked device in association with the navigation space and second tracking data corresponding to the tracked device in association with the virtual space; and generate a notification associated with the one or more discrepancies, perform one or more operations associated with compensating for the one or more discrepancies, or both.

Any of the aspects herein, wherein the virtual space corresponds to a field of view of the imaging device.

Any of the aspects herein, wherein the navigation space and the tracked device are associated with at least one of: an optical tracking system, an acoustic tracking system, an electromagnetic tracking system, a radar tracking system, an inertial measurement unit (IMU) based tracking system, and a computer vision based tracking system.

An imaging system including an imaging device; a tracking system including: a transmission device; and a tracked device; a calibration phantom; a processor; and a memory storing data that, when processed by the processor, cause the processor to: generate a navigation space based on one or more tracking signals emitted by the transmission device; generate a virtual space including at least a portion of the calibration phantom based on one or more images generated by the imaging system, wherein the one or more images are generated in response to one or more signals transmitted by the imaging device; identify a set of coordinates in the virtual space in response to an event in which at least a portion of the tracked device is detected in the one or more images; and calibrate a first coordinate system associated with the virtual space with respect to a second coordinate system associated with the navigation space in response to the event, wherein calibrating the first coordinate system with respect to the second coordinate system is based on the set of coordinates and temporal information associated with the event.

Any of the aspects herein, wherein the set of coordinates are identified in the virtual space in response to at least the portion of the tracked device intersecting a surface of the virtual space at the set of coordinates.

Any of the aspects herein, wherein: calibrating the first coordinate system with respect to the second coordinate system is in response to one or more occurrences of the event; and calibrating the first coordinate system with respect to the second coordinate system is absent pausing a surgical procedure.

Any of the aspects herein, wherein the calibration phantom includes: a water bath; or a tissue phantom included in a body of a subject, wherein the tissue phantom and the subject are associated with a surgical procedure.

Any of the aspects herein, wherein calibrating the first coordinate system with respect to the second coordinate system is based on: beam thickness, beam shape, or both of the one or more signals transmitted by the imaging device; pose information of the portion of the tracked device in association with an intersection between the portion of the tracked device and a plane of the virtual space; and one or more properties of the portion of the tracked device.

A method including: generating a navigation space based on one or more tracking signals; generating a virtual space including at least a portion of a calibration phantom based on one or more images, wherein the one or more images are generated in response to transmitting one or more imaging signals; identifying a set of coordinates in the virtual space in response to an event in which at least a portion of a tracked device in the calibration phantom is detected in the one or more images; and calibrating a first coordinate system associated with the virtual space with respect to a second coordinate system associated with the navigation space in response to the event, wherein calibrating the first coordinate system with respect to the second coordinate system is based on the set of coordinates and temporal information associated with the event.

Any of the aspects herein, wherein the set of coordinates are identified in the virtual space in response to at least the portion of the tracked device intersecting a plane of the virtual space at the set of coordinates.

Any of the aspects herein, wherein: calibrating the first coordinate system with respect to the second coordinate system is in response to one or more occurrences of the event; and calibrating the first coordinate system with respect to the second coordinate system is absent pausing a surgical procedure.

Any of the aspects herein, wherein the calibration phantom includes: a water bath; or a tissue phantom included in a body of a subject, wherein the tissue phantom and the subject are associated with a surgical procedure.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/implementations in combination with any one or more other aspects/features/implementations.

Use of any one or more of the aspects or features as disclosed herein.

It is to be appreciated that any feature described herein can be claimed in combination with any other feature(s) as described herein, regardless of whether the features come from the same described implementation.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Aspects of the present disclosure may take the form of an implementation that is entirely hardware, an implementation that is entirely software (including firmware, resident software, micro-code, etc.) or an implementation combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including, but not limited to, wireless, wireline, optical fiber cable, RF, etc., or any suitable combination of the foregoing.

The terms “determine,” “calculate,” “compute,” and variations thereof, as used herein, are used interchangeably and include any type of methodology, process, mathematical operation or technique.

What is claimed is:

1. A system comprising:
a processor; and
a memory storing instructions that, when executed by the processor, cause the processor to:
generate a navigation space based on one or more tracking signals;
generate a virtual space comprising at least a portion of a calibration phantom based on one or more images, wherein the one or more images are generated in response to one or more signals transmitted by an imaging device;
identify a set of coordinates in the virtual space in response to an event in which at least a portion of a tracked device in the calibration phantom is detected in the one or more images; and
calibrate a first coordinate system associated with the virtual space with respect to a second coordinate system associated with the navigation space in response to the event, wherein calibrating the first coordinate system with respect to the second coordinate system is based on the set of coordinates and temporal information associated with the event.

2. The system of claim 1, wherein the set of coordinates are identified in the virtual space in response to at least the portion of the tracked device intersecting a surface of the virtual space at the set of coordinates.

3. The system of claim 1, wherein:
calibrating the first coordinate system with respect to the second coordinate system is in response to one or more occurrences of the event; and
calibrating the first coordinate system with respect to the second coordinate system is absent pausing a surgical procedure.

4. The system of claim 1, wherein calibrating the first coordinate system with respect to the second coordinate system is based on:
beam thickness, beam shape, or both of the one or more signals transmitted by the imaging device;
pose information of the portion of the tracked device in association with an intersection between the portion of the tracked device and a plane of the virtual space; and
one or more properties of the portion of the tracked device.

5. The system of claim 1, wherein the calibration phantom comprises:
ultrasound conductive material; or
a tissue phantom comprised in a body of a subject, wherein the tissue phantom and the subject are associated with a surgical procedure.

6. The system of claim 1, wherein the instructions are further executable by the processor to:
output guidance information associated with positioning the imaging device, the tracked device, or both in association with calibrating the first coordinate system with respect to the second coordinate system.

7. The system of claim 1, wherein the tracked device is comprised in at least a portion of an instrument, and the instructions are further executable by the processor to:
detect, in the one or more images, one or more landmarks corresponding to at least a portion of the tracked device, wherein calibrating the first coordinate system with respect to the second coordinate system is based on the one or more landmarks.

8. The system of claim 1, wherein calibrating the first coordinate system with respect to the second coordinate system comprises verifying a registration accuracy between the first coordinate system and the second coordinate system.

9. The system of claim 1, wherein the instructions are further executable by the processor to:
detect one or more discrepancies between first tracking data corresponding to the tracked device in association with the navigation space and second tracking data corresponding to the tracked device in association with the virtual space; and
generate a notification associated with the one or more discrepancies, perform one or more operations associated with compensating for the one or more discrepancies, or both.

10. The system of claim 1, wherein the virtual space corresponds to a field of view of the imaging device.

11. The system of claim 1, wherein the navigation space and the tracked device are associated with at least one of: an optical tracking system, an acoustic tracking system, an electromagnetic tracking system, a magnetic tracking system, a radar tracking system, an inertial measurement unit (IMU) based tracking system, and a computer vision based tracking system.

12. A system comprising:
an imaging system comprising an imaging device;
a tracking system comprising:
a transmission device; and
a tracked device;
a calibration phantom;
a processor; and
a memory storing data that, when processed by the processor, cause the processor to:
generate a navigation space based on one or more tracking signals emitted by the transmission device;
generate a virtual space comprising at least a portion of the calibration phantom based on one or more images generated by the imaging system, wherein the one or more images are generated in response to one or more signals transmitted by the imaging device;
identify a set of coordinates in the virtual space in response to an event in which at least a portion of the tracked device is detected in the one or more images; and
calibrate a first coordinate system associated with the virtual space with respect to a second coordinate system associated with the navigation space in response to the event, wherein calibrating the first coordinate system with respect to the second coordinate system is based on the set of coordinates and temporal information associated with the event.

13. The system of claim 12, wherein the set of coordinates are identified in the virtual space in response to at least the portion of the tracked device intersecting a surface of the virtual space at the set of coordinates.

14. The system of claim 12, wherein:
calibrating the first coordinate system with respect to the second coordinate system is in response to one or more occurrences of the event; and
calibrating the first coordinate system with respect to the second coordinate system is absent pausing a surgical procedure.

15. The system of claim 12, wherein the calibration phantom comprises:
ultrasound conductive material; or
a tissue phantom included in a body of a subject, wherein the tissue phantom and the subject are associated with a surgical procedure.

16. The system of claim 12, wherein calibrating the first coordinate system with respect to the second coordinate system is based on:
beam thickness, beam shape, or both of the one or more signals transmitted by the imaging device;
pose information of the portion of the tracked device in association with an intersection between the portion of the tracked device and a plane of the virtual space; and
one or more properties of the portion of the tracked device.

17. A method comprising:
generating a navigation space based on one or more tracking signals;
generating a virtual space comprising at least a portion of a calibration phantom based on one or more images, wherein the one or more images are generated in response to transmitting one or more imaging signals;
identifying a set of coordinates in the virtual space in response to an event in which at least a portion of a tracked device in the calibration phantom is detected in the one or more images; and
calibrating a first coordinate system associated with the virtual space with respect to a second coordinate system associated with the navigation space in response to the event, wherein calibrating the first coordinate system with respect to the second coordinate system is based on the set of coordinates and temporal information associated with the event.

18. The method of claim 17, wherein the set of coordinates are identified in the virtual space in response to at least the portion of the tracked device intersecting a plane of the virtual space at the set of coordinates.

19. The method of claim 17, wherein:
calibrating the first coordinate system with respect to the second coordinate system is in response to one or more occurrences of the event; and
calibrating the first coordinate system with respect to the second coordinate system is absent pausing a surgical procedure.

20. The method of claim 17, wherein the calibration phantom comprises:
ultrasound conductive material; or
a tissue phantom comprised in a body of a subject, wherein the tissue phantom and the subject are associated with a surgical procedure.
